(19) United States
(12) Patent Application Publication
(10) Pub. No.: US 2002/0002336 A1
(43) Pub. Date: Jan. 3, 2002

(54) Optical measurements of bone composition

(76) Inventors: Kevin S. Marchitto, Mt. Eliza (AU); Stephen T. Flock, Mt. Eliza (AU)

Correspondence Address: Benjamin Aaron Adler, ADLER & ASSOCIATES, 8011 Candle Lane, Houston, TX, 77071, US

(21) Appl. No.: 09/765,989

(22) Filed: Jan. 19, 2001

Related U.S. Application Data
(60) Provisional application No. 60/177,508, filed on Jan. 21, 2000.

(51) Int. Cl. A61B 6/00

(52) U.S. Cl. 600/473

(57) ABSTRACT

The present invention provides an non-invasive and inexpensive method and/or device for detecting a disease in a bone or other tissues using an optical fiber based Raman spectrometer by detecting biochemical changes in the bone or the other tissues.

### CROSS-REFERENCE TO RELATED APPLICATION

This application claims benefit of priority of provisional application U.S. Ser. No. 60/177,508, filed Jan. 21, 2000, now abandoned.

### BACKGROUND OF THE INVENTION

1. Field of the Invention

The present invention relates generally to the fields of optical imaging and medical diagnosis. More specifically, the present invention relates to a method and/or device for optical measurements of biochemical compositions in bone or other tissues, therefore, detecting a disease in bone or other tissues.

2. Description of the Related Art

Osteoporosis and related disorders of bone metabolism are a major public health threat, particularly for older individuals. The disease affects more than 25 million Americans and 80% of these individuals are women. A woman's risk of developing a hip fracture, as a consequence of osteoporosis, is equal to her combined risk of developing breast, uterine, and ovarian cancer. Industry studies estimate that the lifetime risk of hip fracture in men approximates the risk of prostate cancer.

Osteoporosis accounts for 1.5 million fractures annually, 300,000 of which are hip fractures. One out of every two women and one in eight men over 50 will have an osteoporosis-related fracture in their lifetime. Depending on what data is included in the statistical cost analysis, the cost of osteoporosis in the United States has been estimated at anywhere from about $3.8 billion to $14 billion each year.

Osteoporosis is referred to as a “silent epidemic” because bone loss occurs without symptoms. In fact, the bone mineral density (BMD) measurement is the only way to make a definitive diagnosis of osteoporosis. Usually, bone disorders are generally suspected only upon circumstantial evidence such as a broken hip, unusual weight loss or pain. For confirmatory diagnoses, bone biopsy can provide some important clinical information not available any other way, however bone densitometry is the diagnostic method of choice.

Bone densitometry can detect osteoporosis before a fracture occurs, predict the patient's chances of fracturing in the future and determine the rate of bone loss, and/or monitor the effects of treatment. A BMD is typically done on the bones in one's spine, hip and/or wrist, the most common sites of fractures due to osteoporosis. BMD is expressed as grams of calcium (the major mineral constituent of bone) per square centimeter (or per cubic centimeter in the case of less available experimental three-dimensional techniques) of bone cross section.

The method of choice for doing modern quantitative densitometry of bone is dual X-ray absorptiometry (DXA). This technique employs x-ray beams of two different wavelengths, one which bone and soft tissue absorb to similar degrees, and a second which bone absorbs much stronger than soft tissue. Based on measurements of the differential transmission from x-ray images, and defining a region-of-interest (ROI) of a particular area, Beer's law can be used to deduce the bone mineral density (BMD) in units of gm/cm². Sometimes the bone mineral content (gm) is quoted, which can be calculated from the product of BMD and the area. One problem with this measurement is that it does not take into account the thickness of the bone through which the x-rays propagate, and so can be in error. Thus, the term “bone density” is actually incorrectly used; true bone density (with units of mass per unit volume) may actually increase with age. In fact, DXA provides a measurement of absorption throughout the entire bone volume imaged. Nevertheless, clinical experience with this technique shows that it is able to explain about 70-75% of the variance in bone strength; presumably the remaining 25-30% can be explained by accumulated burden of fatigue damage, state of bone remodeling, measuring artifacts, etc.

In theory, BMD measurements may be taken on any bone, as osteoporosis is a systemic disease which affects all skeletal sites similarly (albeit not homogeneously). As a matter of practicality, it has been shown that measurements of BMD at the site where fractures are most problematic (hip and lumbar spine) correlate best with the probability of a fracture occurring at that site. Measurements taken at other sites do provide useful clinical information however. For example, measurements at the femoral neck are often used to diagnose and assess treatment of osteopenia (low bone mass).

BMD measurements using DXA have an accuracy of 5-10%. However BMD measurements are relatively expensive and not performed routinely. This is the result of cost and the use of ionizing radiation. Consequently, whole population screening is not practical, even though studies have shown that such an approach, leading to early intervention in the management of osteoporosis, would be beneficial. Accordingly, the National Institutes of Health Consensus Development Conference Statement (Apr. 2-4, 1984) panel has recommended that “Studies (be done) to develop accurate, safe, inexpensive methods for determining the level of risk for osteoporosis in an individual, to establish early diagnosis, and to assess the clinical course of the disease.”

Raman spectroscopy provides significant benefits over other forms of spectroscopy. These include greater depth of penetration of incident light, superior discrimination between organic and inorganic peaks, and minimal sample preparation. Perhaps the most important advantage is that water, which is ubiquitous in tissue, does not produce a Raman signal that overwhelms important organic signals. Rehman and co-workers noted that deproteination of bone is not necessary before obtaining useful spectral information in bone, thus suggesting that in vivo application may be possible.

Although many studies have been done on tissues samples ex vivo using Fourier Transform Infrared Spectroscopy (FTIR), and a few have used Raman spectroscopy (e.g. ref ), there are very few in vivo studies using Raman. This is probably because CCD detectors with the necessary sensitivity for Raman have only recently become available. It is again worthy of mention that perhaps the most important benefit of Raman spectroscopy over FTIR is that the incident light used to probe tissue can penetrate deeply (centimeters, depending on wavelength), whereas FTIR can only be used to probe samples about a few microns deep.

Nonetheless, some studies have utilized infrared spectroscopy measurements to evaluate bone mineral content and these provide useful information. Numerous infrared spectroscopy studies involving measurement of the mineralization, crystalization, and inorganic constituents in bone have been performed. Among the most relevant ones are those discussed below, and a general reference on infrared spectroscopy in tissues is provided (Jackson and Mantsch).

TABLE 1
Some relevent infrared absorption and scattering lines in bone

| | | Wave | |
|---|---|---|---|
| | Spectal | number | |
| Species | Notation | (cm⁻¹) | Comments |
| PO₄³⁻ | λ₁ | 500-600 | |
| phosphate | | | |
| | λ₁ | 940-960 | Raman; very strong |
| | λ₃ | 1017-1070 | Raman; strong |
| | λ₃ | 1180-1200 | |
| CO₃²⁻ | λ₂ | 840-900 | Triplet |
| carbonate | | | |
| | λ₂ | 880 | Raman; weak |
| Amide I | | 1595-1750 | Raman and FTIR; |
| (collagen) | | | very strong. C = O |
| | | | and C − N stretch. |
| Phenyl | | 1005 | Raman |
| peptide | | | |
| groups | | | |

Table 1 shows some of the most relevant absorption lines in bone. The positions of the lines are only approximate as the source of the bone material and the sample preparation can have an effect on the infrared absorption spectrum. Extensive infrared spectrometric measurements have been performed on calcified tissue; some of the most relevant of these are summarized in Table 2, which follows.

TABLE 2
A summary of some of the results of infrared spectrometric studies
on calcified tissue

| | | | PO₄ | CO₃ | Organic | |
|---|---|---|---|---|---|---|
| Author | Date | Sample | (cm⁻¹) | (cm⁻¹) | (cm⁻¹) | Comments |
| Walton | 1 | ox tibia, | 955 | | 1663 | Phosphate |
| et al. | 9 | reconst. | | | | most intense |
| (Raman) | 7 | rat tail | | | | band in |
| | 0 | collagen | | | | spectrum |
| Ohsaki | 1 | human | 567- | | 1005 | organic |
| et al. | 9 | bone | 1017 | | | 1005 |
| (Raman) | 8 | fragments | | | | cm-1 good |
| | 8 | from | | | | internal |
| | | infected | | | | standard; |
| | | ear | | | | FWHM |
| | | | | | | affected by |
| | | | | | | crystallinity; |
| | | | | | | PO₄/1005 |
| | | | | | | less in |
| | | | | | | damaged |
| | | | | | | bone. |
| Rey et al. | 1 | rat, | | 871- | | 878/871 |
| (FTIR) | 9 | chicken | | 878 | | constant |
| | 8 | cow and | | | | with age, |
| | 9 | human | | | | even |
| | | bone | | | | among |
| | | | | | | species |
| Rey et al. | 1 | chicken | | 860- | | 878/811 |
| (FTIR) | 9 | bone | | 878 | | constant |
| | 9 | | | | | with age; |
| | 1 | | | | | 860/871 |
| | | | | | | goes down |
| | | | | | | with age. |
| Rey et al. | 1 | chicken | 900- | 830- | | FWHM |
| (FTIR) | 9 | bone | 1200 | 885 | | broadens |
| | 9 | osteo- | 530- | | | with age |
| | 6 | blasts, | 630 | | | in PO₄ |
| | | native | | | | and CO₃; |
| | | chicken | | | | PO₄ lines |
| | | bone | | | | less |
| | | | | | | prominent |
| | | | | | | with age. |
| Paschalis | 1 | human | 900- | 850- | 1585- | PO₄ and |
| et al. | 9 | osteonal | 1200 | 900 | 1725 | Amide I |
| (FTIR) | 9 | bone | 500- | | | lines more |
| | 6 | | 660 | | | prominent |
| | | | | | | with age, |
| | | | | | | CO₃/(PO₄/ |
| | | | | | | AmideI) |
| | | | | | | decreases |
| | | | | | | with age. |
| Pienkow- | 1 | canine | 900- | 840- | 1595- | PO₄/Amide I |
| ski | 9 | vertebrae | 1180 | 890 | 1750 | and CO₃/ |
| et al. | 9 | | | | | PO₄ |
| (FTIR) | 6 | | | | | change with |
| | | | | | | time in |
| | | | | | | calcitonin |
| | | | | | | treated dogs. |
| Boskey | 1 | osteo- | 900- | 855- | 1585- | mineral to |
| et al. | 9 | calcin | 1200 | 885 | 1720 | matrix |
| (FTIR) | 9 | deficient | | | | and carbo- |
| | 8 | and wild- | | | | nate to |
| | | type; | | | | mineral |
| | | ovariec- | | | | ratios |
| | | tomized | | | | different |
| | | mice | | | | depending |
| | | | | | | on mouse. |

Rey and co-workers measured the environment of carbonate ions in several different animal species as well as humans and osteoblast cell culture, using Fourier Transform Infrared Spectroscopy (FTIR). They observed carbonate bands in the λ₂ band (the B vibrational band where carbonate ions substitute for phosphate ions in their crystallographic locations by ion exchange), one of which is at 871 cm⁻¹, and which is sharp and not interfered with by protein bands. They also measured carbonate bands, the A vibrational band, where carbonate ions substitute for OH⁻ ions in their crystallographic locations by ion exchange, at 878 cm⁻¹, and a shoulder at 866 cm⁻¹ which they hypothesize corresponds to CO₃ in unstable locations. They observed that the ratio of the band intensities of the 878 to 871 absorptions stayed constant at about 0.77 however, the ratio of the 866 to 871 bands decreased as the age of the examined bone increased. In the osteoblast culture, they observed that the carbonate bands at 866 and 879 broaden with aging. They conclude that the bandwidths are a function of the surrounding environment, age of bone and state of mineralization.

Paschalis and co-workers performed measurements of the mineral and organic matrix quantities in osteoporotic human osteonal bone. By integrating the PO₄ bands (900-1200 cm⁻¹) and the Amide I band (1585-1725 cm⁻¹), they determined that the ratio of PO₄/Amide increased from the center to the periphery of osteons; this would be expected based on our knowledge of osteoblast calcium production. The total carbonate quantity was measured by integrating the absorption bands between 850-900 cm⁻¹. They determined that the ratio of the carbonate over the ratio of PO₄/Amide increased with increasing bone age. They further determined that the ratio of the PO₄ absorptions at 1020 to 1030 cm⁻¹ decreased with distance from the osteon center. The conclusion is that all of these ratios can be used to monitor mineral quality in bones.

Pienkowski et al. measured the effects of calcitonin in a dog model using DXA and FTIR. They measured the phosphate (P) content by integrating absorption bands from about 900-1180 cm⁻¹, carbonate (C) content between 840-890 cm⁻¹ and Amide I (A) between 1595-1750 cm⁻¹. The relative mineral content, P/A, and the relative carbonate content, C/P, in the canine vertebrae correlated well with gravimetric ash mineral determination and they were able to see changes in these ratios consistent with measurements of BMD using DXA.

Ohsaki et al. measured Raman spectra in human bone fragments from the middle ear. They measured differences in the full width at half maximum (FWHM) of the phosphate bands in the bones as compared to artificial hydroxyapatite. The integrated phosphate line intensities normalized by the organic line at 1005 cm⁻¹(phenyl peptide groups), which was constant over all samples, were 18-20% less in bones from infected ears versus control bones. Boskey and co-workers looked at the bones of osteocalcin deficient mice, and ovariectomized wild type and knockout mice. They measured a mineral (phosphate) to matrix (Amide I) ratio, and a carbonate to phosphate ratio different in each cohort of animals thus illustrating that bone mineral maturation was different in each.

The prior art is deficient in the lack of non-invasive, portable and inexpensive method and/or device for optical measurements of bone composition, therefore for optical diagnosis of metabolic bone diseases. The present invention fulfills this long-standing need and desire in the art.

### SUMMARY OF THE INVENTION

The present invention demonstrates that biochemical changes in bone, as a consequence of disease processes such as osteomalacia and osteoporosis, can be non-invasively assessed using an optical fiber based Raman spectrometer.

In one embodiment of the present invention, there is provided a method for detecting a bone disease in a test subject, comprising the steps of transmitting radiant energy to surface of skin overlaying a bone in the test subject; detecting radiant energy reflected from the skin surface to obtain Raman spectra, wherein the Raman spectra from the skin surface reflect the spectral information on the bone, which reflects biochemical compositions of the bone; and comparing the biochemical compositions of the test bone with those of a normal bone, wherein if the biochemical compositions of the test bone differ from those of the normal bone, the test subject might have a diseased bone. Such method can also be used for detecting a disease in tissues other than the bone.

In another embodiment of the present invention, there is provided a device for detecting a bone disease, comprising a source for producing radiant energy; an applicator for transmitting radiant energy; a means for detecting reflected radiant energy; and a Raman spectrometer.

Other and further aspects, features, and advantages of the present invention will be apparent from the following description of the presently preferred embodiments of the invention given for the purpose of disclosure.

### DETAILED DESCRIPTION OF THE INVENTION

The present invention provides a non-invasive, portable and inexpensive device for optical measurements of bone composition, therefore for optical diagnosis of metabolic bone diseases. It is demonstrated that biochemical changes in bone, as a consequence of disease processes such as osteomalacia and osteoporosis, can be non-invasively assessed using an optical fiber based Raman spectrometer.

Specifically, the present invention is to construct and test a non-invasive Raman spectrometer for measuring spectra in bone; measure Raman spectra of normal and diseased bone ex vivo; and compare results of measurements using Raman spectroscopy to those made with dual x-ray absorptiometry (DXA) in vivo.

Raman spectroscopy provides a means for optically evaluating changes in bone biochemistry through non-invasive probing of tissues with harmless radiant energy. Recent advances in the development of Raman instrumentation now provide the opportunity to develop inexpensive, highly sensitive devices that provide far greater resolution than any optical methods previously available to the clinician. Raman spectroscopy as used herein has a significant advantage over infrared spectroscopy, such as FTIR, in that the incident light penetrates tissue to a significantly greater extent than in the infrared.

Optical measurements and techniques will provide an opportunity for routine measurement of biochemical changes associated with bone disorders in the clinic. The present devices would greatly improve the frequency of screening for bone disorders in patients at risk, as well as the ease of monitoring existing patients, even in a primary care setting. This is important since early intervention is critical for a successful therapy.

Routine measurements of large populations are currently not practical because devices used to measure bone density are expensive and use potentially harmful x-rays. However, the device described herein is not expected to replace current methods of bone densitometry, but to augment the ability of the physician to screen broader cross-sections of the population.

A question may arise as to whether enough photons will be scattered from the bone tissue to provide useful spectral information. The percentage of photons that undergo Raman scattering is typically less than 1%. And, although near-infrared light is relatively penetrating in tissue, it does not have near the penetrating properties of x-rays.

To model the behavior of near infrared radiant energy in this situation, the Monte Carlo program of Wang and Jacques was applied to calculate light dosimetry. These calculations are intended to be an order-of-magnitude estimate of light penetrance and diffuse reflectance. They provide a first approximation, as they do not precisely model the proposed Raman measurements. Nevertheless, with these data, fiber-optic based reflectance probe can then be designed for maximum efficiency and determine whether any spectral information on bone will be reflected from the surface of the tissue probed.

The optical properties of skin are interpolated from the internet tissue optics site of Jacques and Prahl (University of Oregon Health Science Center, 1998), the (rabbit) muscle values are from Beck et al. The values for bone are taken from Firbank et al. for cortical bone. The results of the calculation provide a diffuse reflectance of 13.2%, and light absorption values of 5.2, 77.9 and 1.0% in the skin, muscle and bone layers respectively. Only about 2.0% of the incident light penetrates to the bone layer, but because of the high albedo of bone in the NIR, about 57% of the radiant energy incident on the bone is reflected. Because, however, some of the reflected light will be absorbed in each of the layers, less than 2% of 57%, or less than 1% of the incident light will be scattered back to the surface of the tissue.

Iso-energy fluence profiles are obtained by convolving the data obtained in the Monte Carlo simulations (this assumed a 1 mm diameter incident beam) (data not shown). They indicate that some incident radiant energy, albeit a small fraction of the incident energy, does penetrate into the bone material. A scoring of the radial and angular dependence of the diffuse reflectance in this situation shows that >99% of the reflected radiant energy occurs within 0.2 mm of the center of the injected beam, and most of the reflection is emitted in a cone, centered on the axis of the incident beam, with a half angle of 30 degrees. These calculations illustrate that most of the light would be reflected right back into the optical fiber used to apply the incident beam (assuming an optical fiber diameter of 1 mm). Therefore the most efficient collection geometry is to use a beam-splitter geometry where the reflected radiant energy is captured by the same optical system as is used to apply the incident energy. The calculations show that a (typical) 1×n fiber applicator (i.e. a single, central fiber carrying the incident beam and an annular array of n-fibers arranged around the one) is not the most efficient. A probe will then be designed with coaxial source and collection optics.

Furthermore, because most of the reflected signal is due to overlying tissue, the effects of the tissue must be considered. Very little Raman scattered energy is reflected from the tissue, thus the most sensitive detector possible must be employed. Note also that the Raman shifted scattered photons, which occur at shifts of about 500-1750 cm⁻¹ (λ=817-910 nm), will not be differentially affected by the overlying tissue they must traverse to escape as tissue optical properties between these two wavelengths are relatively constant.

The safe use of lasers in health-care institutions is governed by the Occupational Health and Safety Administration (OSHA) but the regulations regarding the use of devices on humans which employ lasers, is under the control of the Food and Drug Administration (FDA; 21 CFR 1040.10/11). Both of these organizations take their guidelines for laser safety from the American National Standards Institute (ANSI; ANSI Z136.1-1993/ANSI Z136.3-1996). From these, the maximum permissible (long-term; >10 sec.) can be calculated, exposure to skin of 785 nm radiant energy is 590 mW/cm². Thus, for a laser-irradiated spot on the skin, 1 mm in diameter, only about 18.5 mW of near infrared radiant energy can be used. Note that this value falls far below the harmless energy fluence used to activate drugs used in photodynamic cancer therapy However, as heating effects must be considered in Raman measurements, care will be taken to monitor heating and keep it below 60° C., thus avoiding any protein denaturation or lipid melting.

In one embodiment of the present invention, there is provided a method for detecting a bone disease in a test subject, comprising the steps of transmitting radiant energy to surface of skin overlaying a bone in the test subject; detecting radiant energy reflected from the skin surface to obtain Raman spectra, wherein the Raman spectra from the skin surface reflect the spectral information on the bone, which reflects biochemical compositions of the bone; and comparing the biochemical compositions of the test bone with those of a normal bone, wherein if the biochemical compositions of the test bone differ from those of the normal bone, the test subject might have a diseased bone. Preferably, the radiant energy is transmitted through a fiber-optic based reflectance probe, and the radiant energy reflected from the skin surface is collected by a fiber optic. Still preferably, the radiant energy is near-infrared light having a wavelength range of from about 600 nm to about 1500 nm. The reflected radiant energy is filtered through a long-pass filter, a band-pass filter or a polarization filter. Such method is used for detecting a bone disease, such as osteomalacia, osteoporosis, a bone cancer, or a bone infection.

In another embodiment of the present invention, there is provided a device for detecting a bone disease, comprising a source for producing radiant energy; an applicator for transmitting radiant energy; a means for detecting reflected radiant energy; and a Raman spectrometer. Preferably, the applicator for transmitting radiant energy is a fiber-optic based reflectance probe. Still preferably, the radiant energy is near-infrared light.

The method disclosed herein can also be used for detecting a disease in tissues other than the bone.

The following examples are given for the purpose of illustrating various embodiments of the invention and are not meant to limit the present invention in any fashion.

### EXAMPLE 1

Imaging Spectrograph

A HoloSpec f/1.8i Holographic Imaging Spectrograph (Kaiser Optical Systems, Ann Arbor, Mich.), which includes an integrated pre-filter section and provides extremely high throughput, will be used. The aperture ratio of f/1.8 provides five to twenty times greater collection angle than spectrographs operating at f/4 to f/8. It includes a state-of-the-art holographic SuperNotch-Plus filter, which attenuates Rayleigh scatter by over six orders of magnitude, allows collection of data at Stokes shifts as low as 50 wavenumbers, and allows simultaneous collection of both Stokes and anti-Stokes data.

This low f-number holographic spectrograph has a high-throughput and is well matched optically to accept the input from a cut-end optical fiber, which typically provide a cone-of-light near f/2. The spectrograph can accept multiple inputs in the vertical direction so that multiple spectra can be simultaneously captured. A holographic notch filter (centered at 785 nm; OD 6.0, FWHM 10 nm) manufactured by Kaiser will be positioned in the spectrograph to eliminate the Rayleigh scattered 785 nm light and other ambient light. The device will be such that spectra from 500-1800 cm⁻¹ can be captured without scanning.

### EXAMPLE 2

CCD Detector

The detector will be a liquid nitrogen cooled 1340×400 pixel back illuminated charge-coupled-device (CCD) chip manufactured by Roper Scientific, Inc. (formerly Princeton Instruments and Photometrics, Inc., Trenton, N.J.) (Model LN/CCD-400EB). This device has a dynamic range of 16 bits, an extremely low dark charge of less than 1 electron/pixel/hr at liquid nitrogen temperatures, a quantum efficiency of >30-70% at the relevant wavelengths, and an optical configuration to eliminate etaloning. With the included software, multiple spectra can be integrated on the chip simultaneously. It is expected that light collection times of up to an hour or more can be attained with this device while still producing a signal-to-noise ratio large enough for subsequent deconvolution. This device will be coupled to the spectrograph using a custom-machined adapter.

### EXAMPLE 3

Laser

The laser used to provide the incident radiant energy (Model XC30, SDL, Inc., San Jose, Calif.) emits 300 mW of 785 nm wavelength radiant energy. The output is frequency stablized (<±0.1 nm) and has an optical arrangement to eliminate feedback from the output-coupled optical fiber. The radiant output of the laser (intensity stable to ±0.9%) will be split so that two output fibers can be optically coupled to the fiber optic probes (see Example 4 below) with standard FC connectors.

### EXAMPLE 4

Fiber Optic Probe

The (two) probes are of a proprietary design constructed by InPhotonics (Norwood, Mass.). This device (diam.=12.5 mm) allows for co-axial and simultaneous illumination and collection of Raman scattered photons. The collected signal is reflected by a dichroic mirror through a long-pass filter which transmits only the Stokes scattered light and attenuates the Rayleigh band thus preventing the observation of silica Raman bands that arise in the collection fiber. Raman bands due to the silica in the excitation fiber are eliminated with a band-pass filter. Cross-talk between the excitation and collection fiber are eliminated by separating the two in their own cylindrical housings. The probes are coupled via 1-5 m optical fibers terminating in FC connectors, to the input port of the spectrograph in a way that two spectra can be simultaneously captured. The working distance for these probes is 12 mm, but can be extended to greater than 10 cm with a simple change in focusing optics.

### EXAMPLE 5

Spectral Analysis Software

The software suitable for analyzing the collected spectra (background subtraction, baseline interpolation, line integration, boxcar smoothing, deconvolution) will be GRAMS/32 Spectroscopy Software Suite with IR/Raman application package (Galactic Software, Salem, N.H.).

### EXAMPLE 6

Construction and Calibration of Raman Spectrometer

The Raman spectrometer will be constructed on a portable optical table. Thus it will be suitable for moving into the clinic for ultimate measurements in vivo. The device will be tested and calibrated (in wavelength and intensity) before subjecting animals to the experiments using the HoloLab Series Calibration Accessory (Kaiser Optical Systems, Inc.), which is NIST traceable. A tungsten-halogen source within the accessory provides a NIST traceable calibration of the relative intensity axis. An integrated atomic line source allows wavelength calibration to well established spectral standards. A mounted Raman shift standard allows accurate calibration of the laser operating wavelength without removal of laser notch filters, as well as guaranteeing overall system performance and calibration. Resolution of 4 cm⁻¹ and intensity accuracy of >99% will be the design goals.

### EXAMPLE 7

Specimens

Multiple specimens of isolated femur will be obtained from same-sex, same-age, normal wild type mice and mice with bone defects such as C57BL/6J-Hyp, a model for osteomalacia, or B6C3Fe-ala-Csfm, a model for osteoporosis (Jackson Laboratories, Bar Harbor, Minn.). Both the wild type and model mice weight 25-50 g, and 40 of each are to be used. Samples will be harvested after the sacrifice of animals and cartilage will be removed by scraping with a scalpel blade. The samples will be sealed and stored at −70° C. until use.

The biochemical changes in bone associated with osteomalacia or osteoporosis are still not unambiguously established. Furthermore, since the purpose of the present invention is to develop a device suitable for use in vivo, and such a device has never been used before, in vivo animal experiments will be required before any IRB study approval and FDA device approval could possibly be obtained. The number of animals to be used cannot be calculated statistically at this stage. 40 animals of each strain are proposed; if statistically significant data is obtained with fewer animals, then the entire 40 animals will not be used, thus, animal numbers will be kept to a minimum.

The animals will be anesthetized using ACE-promazine, 0.1 mg/kg i.m. The maintenance dose is 0.05 mg/kg. This will keep the animal safely anesthetized for up to 2 hours. No analgesics will be required, as no surgeries will be done.

Animals used to provide bone material will be euthanized prior to bone harvesting by CO₂ inhalation. This method of euthanasia is consistent with the Panel on Euthanasia of the American Veterinary Medical Association.

Overall, some animals will be anesthetized for up to 2 hours maximum. During anesthesia, the spectral probes will be gently pressed up against the skin overlying a bone (e.g. femur). Spectra will be collected, and the animals will be allowed to recover with care being taken to avoid drug-induced hypothermia. The animals will undergo experimentation on multiple days. Some animals will subsequently be euthanized and will have their bones harvested for in vitro spectral measurements.

### EXAMPLE 8

Raman Spectroscopy

The Raman spectra of the samples will be measured using each probe of the prototype Raman spectrometer.

1) The probe will be positioned 12 mm (i.e. the working distance of the fiber optic probes) from the surface of the thawed bone. Because the 785 nm radiant energy will penetrate the relatively thin bone, a plate of black anodized aluminum will be consistently positioned on the side of the bone opposite the probe.

2) The laser power used will be selected by testing the surface temperature of the bone during irradiation. The surface temperature will be measured using an IR thermometer (Omega Instruments, Inc., Stamford Conn.). The IR thermometer will be held in a lab stand, and a computer will monitor the output (via an RS-232 port). Thus the data from this experiment will provide bone surface temperature in a region-of-interest as a function of time during irradiation. The field-of-view of the thermometer is controllable by positioning the device at a particular distance from the bone. The laser output will be maximized to the point such that surface temperature will be kept below the denaturation and melting points of proteins and lipids, or about 60° C.

3) Collection of spectra will be optimized such that the signal-to-noise (S/N) ratio is maximized allowing suitable for identification of the relevant spectral lines. Literature suggests an approximate collection time of 60 seconds is optimal. 30, 45, 60, 75 and 90 second collection times will be tested until the best S/N ratio is obtained.

4) Measurements will be done on several different parts of the bone by repeating step three at different locations.

5) Measurements (steps 1-4) will be repeated for the second (osteoporosis) mouse model.

### EXAMPLE 9

Analysis

The spectral analysis (deconvolution, baseline restoration, line integration) will be done with GRAMS/32 software. Guidance for the necessary spectral analysis (apodization/deconvolution conditions) will be taken from the literature. Briefly, the spectra will be smoothed by boxcar smoothing. The presence of peaks will be identified by taking a second derivative of the region of interest. The number of downward pointing features is equal to the number of absorbance bands in the original spectrum. If after this it is clear that some bands are not resolved, deconvolution will be done by multiplying the Fourier Transform (FT) of the spectrum with an exponential function, multiplied by a triangular or Bessel apodization, followed by an inverse FT. This process should provide a resolution enhancement (i.e. a reduction in the FWHM) of a factor of 2-4 for S/N ratios of 100:1 to 10,000:1. Note that the peak area is altered, and so deconvolution will not be done when peak areas are to be calculated.

Line intensities will be determined by interpolating a baseline under the relevent line and integrating the line area above the baseline. Centroid positions will be determined by second derivative calculations on the deconvolved spectra. Full-widths-at-half-maximum (FWHM) will be calculated at the 50% intensity point of each line (halfway between the interpolated baseline and peak height).

The lines examined and the ratios taken will be, minimally: (878 cm⁻¹/871 cm⁻¹), (866 cm⁻¹/871 cm⁻¹), PO₄ [900-1200 cm⁻¹[/Amide I[1585 cm⁻¹-1725 cm⁻¹], CO₃ [840 cm⁻¹-900 cm⁻¹], (1020 cm⁻¹/1030 cm⁻¹], and phenyl [1005 cm⁻¹]. Means and standard deviations of intensity ratios, centroid positions, and FWHM from multiple samples (>10) will be determined. The data obtained with the knockout and wild-type mice will be compared (within each strain) using an unpaired two-tailed t-test. Any differences at a significance level of better than 0.05 will be considered to be proof of true spectral differences.

It is expected that osteomalacia results in an alteration of the biochemistry of bones more significantly than osteoporosis.

### EXAMPLE 10

Comparison of Measurements Using Raman Spectroscopy to Those Made with DXA in vivo

The spectral changes associated with osteomalacia and osteoporosis in the mouse models will be established as discussed above. In this experiment, the mice will be distributed into four groups (C57BL/6J-Hyp, C57 wild-type, B6C3Fe-ala-Csfm, and B6C3 wild-type) and coded so that their medical status is unknown, except to principle investigator.

Mice will first be anesthetized, positioned in a restraint to eliminate movement, and DXA measurements will be done on the femur in the anterior-posterior plane under the guidance of our medical consultant. Briefly, the x-ray beam will be positioned perpendicular to the longitudinal axis of the bone. A petri dish filled with a 2.5 cm deep layer of Ringer's solution and placed on top of the PMMA x-ray beam attenuating blocks, as per manufacturer's instructions (Hologic Inc., Waltham, Mass.) for imaging small specimens. The bone density will be determined at three different longitudinal positions in the bone and expressed in units consistent with humans (gm/cm²). The technician doing the BMD analysis with the x-rays will be blinded to the strain of mouse. In the first experiment, 4 mice of each strain and wild-type control, will be studied. The means and standard deviations of the measurements in each of the four groups will be determined

Raman spectral measurements will then be made. One fiber-optic probe will be positioned at 12 mm over the femur of an anesthetized and restrained mouse. The second fiber-optic probe will be positioned 12 mm over the tissue proximal to the femur, but not over the bone. A plate of black anodized aluminum will be consistently positioned on the side of the femur opposite the probe.

It is probable that significantly longer integration times will be needed to compensate for overlying tissue effects that shield the bone from the optical probe. In this case, to compensate, the mice will initially be probed for up to one hour. Subsequently, the time will be reduced in five-minute increments until a second derivative calculation on the spectra fails to show evidence of PO₄ or CO₃ bands.

### EXAMPLE 11

Evaluation of Data

Once spectra are gathered, the pre-analysis spectral will be performed for the appropriate spectral lines. If the spectral changes resulting from osteoporosis and osteomalacia involve changes in FWHM or positions of the mineral components of phosphate or carbonate, analysis will be followed (see Example 12). If the data collected is different than that found during the experiments of Example 9, then the ability will be tested to remove the effects of overlying t issue by one of following two ways:

(1) The spectra will be smoothed and normalized to each other by matching the baselines interpolated under the entire spectrum. Next, the spectrum obtained with the probe that was not positioned over the bone will be subtracted from the spectrum taken with the probe over the bone. If, after this subtraction, the only features visible are those of the mineral components, then this method will be considered a feasible way to remove the effects of overlying tissue.

(2) Alternatively, the method of removing the effects of overlying tissue will be tested whereby the organic spectral lines are ignored and the inorganic signals are focused on while using the invariant internal standard which is phenyl [1005 cm⁻¹] for normalization of each spectra. Here, the ratio of the intensity of each spectral feature to that of phenyl will be calculated.

### EXAMPLE 12

Analysis

Means and standard deviations from the data (line positions, FWHM, ratios, intensities) will be calculated and tabulated along with the BMD measurements. The comparison of these data will be done using Fisher's (two-tailed) exact test, which will test the null hypothesis that the values obtained with Raman spectroscopy and BMD do not correlate. The probability of rejecting the null hypothesis when it is true will be 0.05. Correlation of BMD and any of the infrared spectral data will be considered proof that the device and concept works.

Discussion

The long-term objective is to develop an inexpensive, reliable, non-invasive device that can be used in most general clinics for routine screening of biochemical changes in bone that result from disease processes, specifically osteoporosis. Phase II research would entail development of prototypical systems that could be used in a commercial setting These units would be used first to evaluate bone density in human specimens followed by actual clinical studies of osteoporotic patients. These studies will compare determinations made by DXA to those obtained by the optical systems.

Human measurements using the proposed device will be proposed in a subsequent SBIR Phase II application, and all laser safety precautions will be adhered to. However, based on literature where near-infrared radiant energy is used in photodynamic therapy, hyperthermia and/or photocoagulation, a radiant energy of several hundered milliwatts of 785 nm radiant energy can be used safely in humans with no side-effects or significant warming. Furthermore, one can use larger spot-sizes and/or multiple beams/probes to reduce the energy fluence at any one site to levels below the threshold for side-effects.

The present device also has utility in other applications. Biochemical changes occur during all disease processes, and so it is conceivable that this device could prove to be useful in diagnosing other diseases such as cancer or infections, and for monitoring treatment by detecting blood analytes or drug concentrations.

The following references were cited herein.

1. Osteoporosis. NIH Consens. Dev. Conf. Consens. Statement Online Apr. 2-4; 1984 5(3):1-6.

2. Ibid, 1984.

3. Osteoporosis and Related Bone Diseases˜National Resource Center Washington D.C. 1998. (web site www.osteo.org, data cited Jun. 22, 1998).

4. Parfitt A. M. J. Bone Mineral Research 13, 1998 (preprint).

5. Ryan P. Sem. Nucl. Med. 27, 197-209, 1997.

6. Hans D, et al., Ballieres Clin. Rheumatol. 11, 495-515, 1997.

7. Ryan, Ibid, 1997. Hans et al., Ibid, 1997.

8. R. Soames (ed.). Skeletal Systems. In: Gray's Anatomy, L. Bannister, et al. (eds.). Churchill Livingstone: NY, 1995.

9. Krane S., et al., Metabolic Bone Disease. In: Harrison's Principles of Internal Medicine. (A. Fauci et al., eds.) McGraw-Hill:New York. 1998.

10. Eicher E., et al., Proc. Natl. Acad. Sci. 73, 4667-4671, 1976.

11. Rehman I., et al., J. Biomed. Materials Res. 29, 1287-1294, 1995.

12. Gniadecka M et al. J. Invest. Derm. 111, 1129-1133, 1998.

13. Jackson M., et al., Biomedical Infrared Spectroscopy. In: Infrared Spectroscopy of Biomolecules. Mantsch H and Chapman D (eds.), Wiley-Liss, NY. 1995.

14. Walton A., et al., Calc. Tiss. Res. 6, 162-167, 1970.

15. Rey C., et al., Calcif. Tissue Int. 45, 157-164, 1989.

16. Rey C., et al., Calcif. Tissue Int. 49, 251-258, 1991.

17. Rey C., et al., Conn. Tiss. Res. 35, 343-349, 1996.

18. Walton et al., ibid, 1970.

19. Paschalis E., et al., Calcif. Tissue Int. 59, 480-487, 1996.

20. Puzas J E. The Osteoblast. Chapter 3 In: Primer on the Metabolic Bone Diseases and Disorders of Mineral Metabolism-2^{nd} ed. Favus M J et al. (eds.) Lippincott-Raven:Philadelphia, 1993.

21. Pienkowski D., et al., J. Bone Mineral Res. 12, 1936-1943, 1997.

22. Ohsaki K, et al. Am. J. Otolaryn. 9, 117-126, 1988.

23. Boskey et al. Bone 23, 187-196, 1998.

24. Wang L., et al., Downloaded from Oregon Medical Laser Center, 1998, www.ee.ogi.edu/omlc/

25. Beck et al. Phys. Med. Biol. 42, 2255-2261, 1997.

26. Firbank M., et al., Phys. Med. Biol. 38, 503-510, 1993

27. Eicher et al. Ibid, 1976.

28. Yoshibo H, et al. Nature 345, 442-444, 1990.

29. Duck, F A. Physical Properties of Tissue. A Comprehensive Reference Book. Academic Press: NY, 1990.

30. Camacho N P et al. Bone 17, 217-278, 1995.

31. Smith B C. Fundamentals of Fourier Transform Infrared Spectroscopy. CRC Press:Boca Raton, 1996.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method for detecting a bone disease in a test subject, comprising the steps of:
transmitting radiant energy to surface of skin overlaying a bone in the test subject;
detecting radiant energy reflected from said skin surface to obtain Raman spectra, wherein the Raman spectra from the skin surface reflect the spectral information on the bone, wherein said spectral information on the bone reflects biochemical compositions of said bone; and
comparing the biochemical compositions of the test bone with those of a normal bone, wherein if the biochemical compositions of the test bone differ from those of the normal bone, the test subject might have a diseased bone.

2. The method of claim 1, wherein the radiant energy is transmitted through a fiber-optic based reflectance probe.

3. The method of claim 1, wherein the radiant energy reflected from the skin surface is collected by a fiber optic.

4. The method of claim 1, wherein said radiant energy is near-infrared light.

5. The method of claim 4, wherein said near-infrared light has a wavelength range of from about 600 nm to about 1500 nm.

6. The method of claim 1, wherein said reflected radiant energy is filtered through a long-pass filter, a band-pass filter or a polarization filter.

7. The method of claim 1, wherein said bone disease is selected from the group consisting of osteomalacia, osteoporosis, a bone cancer, and a bone infection.

8. A device for detecting a bone disease, comprising:
a source for producing radiant energy;
an applicator for transmitting radiant energy;
a means for detecting reflected radiant energy; and
a Raman spectrometer.

9. The device of claim 8, wherein said applicator for transmitting radiant energy is a fiber-optic based reflectance probe.

10. The device of claim 8, wherein said radiant energy is near-infrared light.

11. A method for detecting a disease in a test subject, comprising the steps of:
transmitting radiant energy to surface of skin overlaying a tissue of interest in the test subject;
detecting radiant energy reflected from said skin surface to obtain Raman spectra, wherein the Raman spectra from the skin surface reflect the spectral information on the tissue, wherein said spectral information on the tissue reflects biochemical compositions of said tissue;
comparing the biochemical compositions of the test tissue with those of a normal tissue, wherein if the biochemical compositions of the test tissue differ from those of the normal tissue, the test subject might have a disease.

12. The method of claim 11, wherein said disease is a cancer or an infection.
